(19) United States
(12) Patent Application Publication
(10) Pub. No.: US 2006/0210549 A1
(43) Pub. Date: Sep. 21, 2006

(54) Controlled release liposomes and methods of use

(76) Inventors: Devendra K. Srivastava, Fargo, ND (US); Sanku Mallik, Winter Springs, FL (US)

Correspondence Address: MUETING, RAASCH & GEBHARDT, P.A., P.O. BOX 581415, MINNEAPOLIS, MN, 55458, US

(21) Appl. No.: 11/223,225

(22) Filed: Sep. 9, 2005

Related U.S. Application Data
(60) Provisional application No. 60/609,124, filed on Sep. 10, 2004.

(51) Int. Cl. A61K 38/48; A61K 9/127

(52) U.S. Cl. 424/94.63; 424/450

(57) ABSTRACT

The present invention provides liposomes that include a trigger polypeptide, a lipid layer, and a compartment surrounded by the lipid layer and methods of using the liposomes.

This application claims the benefit of U.S. Provisional Application Ser. No. 60/609,124, filed Sep. 10, 2004, which is incorporated by reference herein.

### GOVERNMENT FUNDING

The present invention was made with government support under Grant No. 1R15 DK56681-01A1 and 1P20 RR 1566-01, awarded by the National Institutes of Health. The Government has certain rights in this invention.

### BACKGROUND

Various drug carriers (e.g., liposomes, polymers, micro-spheres, antibody-drug conjugates) have been developed to alter the bio-distribution and pharmacokinetic properties of drug molecules. Among such carriers, liposomes offer several advantages as clinical drug delivery vehicles, and at present, there are 13 liposome-mediated drug delivery systems approved for the treatment of a variety of human diseases (e.g., breast cancer, ovarian cancer, meningitis, fungal infections, leukaemia, and others) (Torchilin, Nat. Rev. Drug Discovery, 2005, 4, 145-160). In addition, the liposome mediated delivery of about 30 other small molecule drugs, DNA fragments, and diagnostic compounds are currently at different stages of clinical trials (Felnerova et al., Curr. Opin. Biotechnol., 2004, 15, 518-529). In recent years, liposomes have also been tested as vehicles for gene delivery in approaches for treating human diseases (M. C. de Lima et al., Current Medicinal Chemistry, 2003, 10, 1221-1231; C. Nicolazzi et al., Current Medicinal Chemistry, 2003, 10, 1263-1277; V. Kumar et al., A. Current Medicinal Chemistry, 2003, 10, 1297-1306; S. Li et al., Liposomes: Rational Design; Janoff, A. S. (Ed.), Marcel Dekker, New York, 1999, pp. 89-124).

Many drugs, especially the anti-cancer drugs, cause severe and sometimes life-threatening side effects. Liposomes have been used to reduce these undesirable side effects. Liposomal doxorubicin and other anthracyclin formulations have been approved for clinical use (A. Gabizon et al., Liposomes: Rational Design; Janoff, A. S. (Ed.), Marcel Dekker, New York, 1999, pp. 343-362). These formulations show many advantages, viz., prolonged circulation times, protection of key organs against toxicity, and accumulation of liposome-encapsulated drugs in solid tumors (A. Gabizon et al., Liposomes: Rational Design; Janoff, A. S. (Ed.), Marcel Dekker, New York, 1999, pp. 343-362). In order to achieve selective targeting, recognition moieties are attached to the outer surface of the liposomes. The targeting group can be an antibody, (G. A. Koning et al., Cancer Detection Prevention 2002, 26, 299-307; U. B. Nielson et al., Biochim. Biophys. Acta, 2002, 1591, 109-118; C. Turner et al., S. J. Liposome Res. 2002, 12, 45-50; R. Banerjee, J. Biomaterials Applications, 2001, 16, 3-21)(K. Maruyama et al., Adv. Drug Delivery Rev., 1999, 40, 89-102; N. Oku, Adv. Drug Delivery Rev., 1999, 40, 63-73; D. D. Lasic, Tibtech, 1998, 16, 307-321) a peptide, (L. Zhang, et al., J. Biol. Chem., 2001, 276, 35714-35722; K. Vogel et al., J. Am. Chem. Soc., 1996, 118, 1581-1586) or small molecules, (A. Gabizon et al., S. Adv. Drug. Delv. Rev., 2004, 56, 1177-1192; C. P. Leamon et al., Adv. Drug. Delv. Rev., 2004, 56, 1127-1141) which target specific receptors.

Usually upon targeting, the encapsulated drugs are released passively to the selected tissue sites. This is based on the transport property of the molecules across the lipid bilayers of liposomes. Triggered release of drugs and labeled molecules from liposomes has been recognized to be an attractive therapeutic approach. In this approach of drug delivery, the liposomes, particularly non-polymerizable liposomes, which are most frequently used as the drug delivery vehicles, do not release contents until the membranes are destabilized by the external agents (trigger). The trigger can be a change in pH, (M. F. Francis et al., Biomacromolecules, 2001, 2, 741-749; D. C. Drummond et al., Progress Lipid Res., 2000, 39, 409-460; M. J. Turke et al., Biochim. Biophys. Acta., 2002, 1559, 56-68; J. A. Boomer et al., Langmuir, 2003, 19, 6408-6415) mechanical stress,(N. Karoonuthaisiri et al., Colloids and Surfaces, B: Biointerfaces, 2003, 27, 365-375; C. Mader et al., Biochim. Biophys. Acta, 1999, 1418, 106-116; V. S. Trubetskoy, J. Controlled Release, 1998, 59, 13-19) metal ions (S. C. Davis et al., Bioconj. Chem., 1998, 9, 783-792), temperature (S. B. Tiwari, J. Drug Targeting, 2002, 10, 585-591; P. Chandaroy et al., J. Controlled Release, 2001, 76, 27-37; H. Hayashi et al., Bioconj. Chem., 1999, 10, 412-418), light (Z. Li et al., Langmuir, 2003, 19, 6381-6391; Y. Wan et al., J. Am. Chem. Soc., 2002, 124, 5610-5611; C. R. Miller et al., FEBS Letters, 2000, 467, 52-5; M. Babincova et al., J. Magnetism Magnetic Mater., 1999, 194, 163-166), or enzymes such as elastase (P. Meers, Adv. Drug Deliv. Reviews, 2001, 53, 265-272), alkaline phosphatase (L. Zhang et al., J. Biol. Chem., 2001, 276, 35714-35722; K. Vogel et al., J. Am. Chem. Soc., 1996, 118, 1581-1586), trypsin (C. C. Pak et al., Biochim. Biophys. Acta, 1998, 1372, 13-27), and phospholipase A₂ (N. Seki, Polym. Bull., 1985, 13, 489-492; S. Takeoka, Macromolecules, 1991, 24, 1279-1283; H. Ringsdorf, Physical Chemistry of Biological Interfaces, Baszkin, A.; Norde, W. (Ed), Marcell Dekker, New York, N.Y., 2000, pp. 243-282; H. Ringsdorf et al., Angew. Chem. Intl. Ed. Engl., 1988, 27, 114-158) (L. Hu et al., Biochem. Biophys. Res. Commun., 1998, 141, 973-978; J. Davidsen et al., Int. J. Pharm., 2001, 214, 67-69; J. Davidsen et al., Biochim. Biophys. Acta, 2003, 1609, 95-101). Conformational changes of peptides, induced by the change in pH, have also been used to facilitate the content release from liposomes (M. J. Turke et al., Biochim. Biophys. Acta., 2002, 1559, 56-68; J. A. Boomer et al., Langmuir, 2003, 19, 6408-6415). Two agents (light and enzymes; light and pH change) acting in sequence have been used as the liposomal triggers (O. V. Gerasimov et al., Advanced Drug Delivery Reviews, 1999, 38, 317-338; N. J. Wymeret al., Bioconj. Chem., 1998, 9, 305-308). When the liposomes are conjugated to an antibody (M. F. Francis et al., Biomacromolecules, 2001, 2, 741-749; D. C. Drummond et al., Progress Lipid Res., 2000, 39, 409-460; M. J. Turke et al., Biochim. Biophys. Acta., 2002, 1559, 56-68; J. A. Boomer et al., Langmuir, 2003, 19, 6408-6415) or a suitable ligand (M. J. Turke et al., Biochim. Biophys. Acta., 2002, 1559, 56-68; L. Zhang et al., J. Biol. Chem., 2001, 276, 35714-35722; K. Vogel et al., J. Am. Chem. Soc., 1996, 118, 1581-1586), both active targeting and triggered release can be achieved at the site of choice.

Hybrid liposomes polymerized with domains of non-polymerizable lipids have been used as the carriers when slow and controlled release of the entrapped molecules (dyes) are required (M. A. Markowitz et al., Diagnostic Biosensor Polymers, American Chemical Society, Washington, D.C., 1994, pp. 264-274). In hybrid liposomes, the non-polymerizable lipids phase-separate, during the polymerization process, forming separate lipid domains (N. Seki et al., Polym. Bull., 1985, 13,489-492; S. Takeoka et al., Macromolecules, 1991, 24, 1279-1283; H. Ringsdorf, Physical Chemistry of Biological Interfaces, Baszkin, A.; Norde, W. (Ed), Marcell Dekker, New York, N.Y., 2000, pp. 243-282; H. Ringsdorf et al., Angew. Chem. Intl. Ed. Engl., 1988, 27, 114-158). The amount of non-polymerizable lipids can be adjusted to control the rate of release of the entrapped molecules (S. Takeoka et al., Macromolecules, 1991, 24, 1279-1283). Hybrid liposomes can be selectively opened at the non-polymerized domains (“uncorking” of the liposomes) using a detergent, a suitable chemical (reducing or oxiding agents) or an enzyme (e.g., PLA₂) (H. Ringsdorf, Physical Chemistry of Biological Interfaces, Baszkin, A.; Norde, W. (Ed), Marcell Dekker, New York, N.Y., 2000, pp. 243-282). The resultant liposomes with “holes” retain the spherical structure and rapidly release their contents to the outside media (H. Ringsdorf, Physical Chemistry of Biological Interfaces, Baszkin, A.; Norde, W. (Ed), Marcell Dekker, New York, N.Y., 2000, pp. 243-282).

There are reports in the literature of photo-initiated destabilization of the hybrid liposomes (A. Mueller et al., Macromolecules, 2000, 33, 4799-4804; B. Bondurant et al., J. Am. Chem. Soc., 1998, 120, 13541-13542; D. E. Bennett et al., Biochemistry, 1995, 34, 3102-3113). These liposomes are composed of polymerizable lipids (containing conjugated dienes at the end of the hydrophobic chains) and saturated lipids. The liposomes rapidly release their contents, when exposed to the UV light, during the polymerization process (T. Spratt et al., Biochim. Biophys. Acta, 2003, 1611, 35-43). The literature reports indicate that the hybrid liposomes are either stabilized or destabilized by polymerizations, depending on the structures of the polymerizable lipids (A. Mueller et al., Chem. Rev., 2002, 102, 727-757).

Unpolymerized as well as polymerized liposomes, after intravenous administration, are rapidly recognized by the phagocytic cells of the reticuloendethelial system. As a result, the liposomes are removed from blood stream and accumulate mostly in liver and spleen within a few minutes to a few hours after injection (D. Ppahadjopous et al., Liposomes: Rational Design, Janoff, A. S. (Ed.), Marcel Dekker, New York, 1999, pp. 1-12). In order to promote long circulation times to liposomes, small amounts (<10%) of polymerizable diacyl phosphatidyl inositol has been incorporated into liposomes (D. Ppahadjopous et al., Liposomes: Rational Design, Janoff, A. S. (Ed.), Marcel Dekker, New York, 1999, pp. 1-12). Incorporation of polyethylene glycol conjugated lipids in the liposomes (stealth liposomes) is an alternative strategy to achieve long circulation times (T. Ishida et al., Biosciences Reports, 2002, 22, 197-224; M. C. Woodle, Long circulating liposomes: Old drugs, new therapies, Strom, G. (Ed.); Springer, Berlin, Germany, 1998).

Unpolymerized liposomes are typically not stable in the gastro-intestinal tract; hence, most of the studies on liposomal delivery rely on the intravenous administration of the drug formulations. However, polymerized liposomes maintain their integrity in the GI tract, and a portion of the administered dose (<10%) gets transported into the systemic circulation (J. Rogers et al., Critical Rev. Therapeutic Drug Carrier Sys., 1998, 16, 421-480). Blood vessels of tumors are inherently leaky due to wider inter-endothelial junctions, large number of fenestrae and discontinuous (or absent) basement membranes (H. F. Dvorak et al., Am. J. Pathol., 1988, 133, 95-109). The openings can be up to 400 nm in diameter. Due to such an increase in vascular permeability, liposomes (of diameter 100 nm or less) are known to accumulate in soft or even in solid tumors (K. Maruyama et al., Adv. Drug Delivery Rev., 1999, 40, 89-102; N. Oku, Adv. Drug Delivery Rev., 1999, 40, 63-73; D. D. Lasic, Tibtech, 1998, 16, 307-321).

Of five major classes of ECM degrading enzymes (viz., cysteine proteases, aspartic proteases, serine proteases, and metalloproteases,), matrix metalloproteases (MMPs) have been implicated in several diseases. Based on the structural features (including the amino acid sequences, domain organizations), 26 different types of MMPs have been recognized in human tissues, which fall into five major classes: (i) collagenases, (ii) gelatinases, (iii) stromelysins and stromelysin like MMPs, (iv) matrilysins, (v) membrane type MMPs, and (vi) other MMPs (viz., MMP-20, MMP-23, and MMP-28) (M. Whittaker et al., Chem. Rev., 1999, 99, 2735-2776; G. Murphy et al., Methods Enzymol., 1995, 248, 470-484; R. Kiyama et al., J. Med. Chem., 1999, 42, 1723-1738). Although many of these MMPs have been implicated in different types of human diseases, gelatinase-A (MMP-2) and gelatinase-B (MMP-9) have been widely recognized to be involved in the progression and metastasis in most of the human tumors. Gelatinase-A and -B have been found to be overexpressed in breast tumors, (M. Polette et al., Virchows Arch Int. J. Pathol., 1994, 424, 641-645; K. Dalberg et al., World J Surg., 2000, 24, 334-340; R. Hanemaaijer et al., Int J Cancer, 2000, 86, 204-207) colorectal tumors, (S. Papadopoulou et al., Tumour Biol., 2001, 22, 383-9; J P Segain et al., J. Cancer Res., 1996, 56, 5506-12) lung tumors, (M. Tokuraku et al., Int J Cancer., 1995, 64, 355-359; H. Nagawa et al., S. Jap. J. Cancer Res., 1994, 85, 934-938) prostate tumors (G. Sehgal et al., Am. J. Pathol., 1998, 152, 591-596), pancreatic tumors (T. Koshiba et al., Surg Today., 1997, 27, 302-304; T M Gress et al., Int J Cancer., 1995, 62, 407-413), and ovarian tumors (T N Young et al., Gynecol Oncol., 1996, 62, 89-99). In fact, the initial discovery of the involvement of MMPs in melanoma cancer and metastasis were ascribed to be due to the overexpression of gelatinase-A and -B (V. Kahari et al., Exp. Dermatol., 1997, 6, 199-213; U. Saarialho-K, Arch. Dermatol., 1998, 294, S47-S54; H. Nagase et al., J. Biol. Chem., 1999, 274, 21491-21494; E. Kerkela et al., Exp. Dermatol., 2003, 12, 109-125; A. R. Nelson et al., J. Clin. Oncol., 2000, 18, 1135-1149; L. A. Liotta et al., Nature, 1980, 284, 67-68).

Aside from the roles of gelatinase-A and -B in tumorigenesis and metastasis in different human tissues, these enzymes have also been found to be involved in other human diseases, such as gouty arthritis (M S Hsieh et al., J Cell Biochem., 2003, 89, 791-799), inflammatory bowel disease (ulcerative colitis) (E. Pirila et al., Dig Dis Sci., 2003, 48, 93-98), abdominal aortic aneurysms (R. Pyo et al., J Clin Invest., 2000, 105, 1641-1649), quiescent Crohn's Disease (A E Kossakowska et al., Ann N Y Acad Sci., 1999, 878, 578-580), glaucoma (C. Kee et al., J Glaucoma., 1999 8, 51-55), and sunlight induced premature skin aging (G J Fisher et al., Curr Opin Rheumatol., 2002, 14, 723-726). Evidently, gelatinase-A and -B exhibit one of the most diverse pathogenic roles, and consequently involved in causing a variety of human diseases, as compared to many other enzymes in the physiological system.

### SUMMARY OF THE INVENTION

The present invention provides a liposome having a trigger polypeptide, a lipid layer, and a compartment surrounded by the lipid layer, wherein the lipid layer includes saturated lipids and unsaturated lipids, a plurality of the saturated lipids include a trigger polypeptide, and wherein three trigger polypeptides form a triple helix. The unsaturated lipids may be polymerized. The triggering polypeptide may include an amino acid repeat region, and the amino acid repeat region may include (GPX)n, wherein X is 4-hydroxyproline, proline, or a homolog thereof, and n is at least 3. The triggering polypeptide may include a peptide bond that is cleaved by a gelatinase-A or a gelatinase-B. The compartment may include a compound such as, for instance, an inhibitor of gelatinase-A, gelatinase-B, or the combination thereof. The present invention also includes a composition that includes the liposome and a pharmaceutically acceptable carrier.

The present invention also provides a method for inhibiting activity of an enzyme, including providing a liposome having a trigger polypeptide present on the surface of the liposome, a lipid layer, and a compartment surrounded by the lipid layer, wherein the trigger polypeptide includes a peptide bond that is cleaved by a first enzyme, wherein three trigger polypeptides form a triple helix, and wherein the compartment includes an inhibitor of a second enzyme. The method further includes exposing the liposome to the enzyme, wherein the first enzyme cleaves the peptide bond and the liposome releases the inhibitor, and wherein the inhibitor inhibits the activity of the second enzyme. The first and second enzymes may be present in vivo, and the first and second enzymes may be the same enzyme or different enzymes.

Further provided by the present invention is a method for treating a disease. The method includes administering to a patient having or at risk of having a disease an effective amount of a composition, and decreasing a symptom of the disease. The composition includes a liposome, wherein the liposome has a targeting polypeptide present on the surface of the liposome, a lipid layer, and a compartment surrounded by the lipid layer, wherein the targeting polypeptide includes a peptide bond that is cleaved by an enzyme, wherein three trigger polypeptides form a triple helix, and wherein the compartment comprises a compound. The enzyme may be gelatinase-A or gelatinasae-B, and the compound may be an inhibitor of the enzyme.

Also provided by the present invention is a method for detecting an enzyme. The method includes administering to a patient an effective amount of a composition comprising a liposome, wherein the liposome has a targeting polypeptide present on the surface of the liposome, a lipid layer, and a compartment surrounded by the lipid layer, wherein the targeting polypeptide includes a peptide bond that is cleaved by an enzyme, wherein three trigger polypeptides form a triple helix, and wherein the compartment comprises an imaging compound, and detecting the presence of the imaging compound in the patient. The imaging compound may be, for instance, a magnetic resonance contrast agents, a fluorescent dye, gadolinium, or magnetic particles.

Unless otherwise specified, “a,” “an,” “the,” and “at least one” are used interchangeably and mean one or more than one. The terms “comprises” and variations thereof do not have a limiting meaning where these terms appear in the description and claims.

### BRIEF DESCRIPTION OF THE FIGURES

FIG. 1. Temperature dependent CD Spectra of LP1. [LP1]=1 mg/mL in 10 mM phosphate buffer, pH 4.0. The peptide solution was stored at 4° C. for 12 hours before recording the spectra.

FIG. 2. The HPLC elution profile of the lipo-peptide LP1 after incubation with MMP-9 for 2 hours is shown in 2A. The HPLC elution profiles of P1 and LP1 after incubation with trypsin are shown in 2B. For clarity, the elution profile of LP1 is plotted with an offset.

FIG. 3. Increase in fluorescence intensity due to the release of carboxyfluorescein is shown. MMP-9 released 55% of the encapsulated dye. No significant release was observed without any enzyme or in the presence of trypsin. The diamonds indicate the release profile from DSPC liposomes (containing no LP1) in the presence of MMP-9.

FIG. 4. Structures of the lipids incorporating an o-nitrobenzyl group and their syntheses.

FIG. 5. Time dependent spectral changes upon irradiation of lipid 1 at 365 nm. The spectra were recorded during 5 minutes of irradiation in 30 second intervals. The insert shows the time slice of the spectral changes at 315 nm. The solid smooth line is the best fit of the data for a single exponential rate equation, with a rate constant of 0.43 min⁻¹.

FIG. 6. The structures of the products after photolysis of lipid 1.

FIG. 7. Kinetics of release of 6-carboxyfluorescein upon irradiation of liposomes incorporating lipid 1 at 365 nm (solid circle). The solid squares represent the control experiment. The solid smooth line is the best fit of the data for a “two-step” liposomal uncorking according to eqn (1), for the k₁ and k₂ values of 0.246 and 0.039 min⁻¹, respectively.

FIG. 8. Excitation and emission spectra of a solution of 6-carboxyfluorescein in HEPES (25 mM, pH=8.0) buffer. Parameters: [dye]=50 μM, slit widths for excitation and emission monochromators, 5 nm. Excitation spectrum was recorded with emission monochromator at 518 nm; for the emission spectrum the excitation wavelength was 480 nm.

FIG. 9. Time dependent increase in fluorescence emission intensity at 518 nm (lex=495) upon irradiation of 6-carboxyfluorescein encapsulated liposomes at 365 nm. The spectra were recorded during 180 minutes of irradiation, in 10 minute intervals.

### DETAILED DESCRIPTION OF PREFERRED EMBODIMENTS OF THE INVENTION

The present invention provides liposomes that can release their contents under specific conditions. A liposome of the present invention includes a lipid layer, a compartment surrounded by the lipid layer, and a triggering polypeptide. The lipid layer of a liposome may be a bilayer (also referred to as unilamellar). The lipid layer typically includes a saturated lipid and an unsaturated lipid, thus, the liposomes of the present invention are also referred to herein as “hybrid liposomes.”

Unsaturated lipids useful herein are typically polymerizable, and can be used to make a polymerized liposome. As used herein, a “polymerizable lipid” is a lipid that can be covalently bound to other lipids having the same or similar structure. A “polymerized liposome” is a liposome made up of at least one type of polymerizable lipid in which some, most, or all of the polymerizable lipids are covalently bound to each other by intermolecular-interactions. The phospholipids can be bound together within a single layer of the phospholipid bilayer (the leaflets) and/or bound together between the two layers of the bilayer. Preferably, the phospholipids are bound together within a leaflet. As used herein, the term “leaflets” is defined as a single layer of phospholipids in the bilayer forming the liposome. Unsaturated lipids can be polymerized by methods routine in the art, including, for instance, ultraviolet irradiation or heat, preferably, ultraviolet irradiation.

An unsaturated lipid includes a hydrophobic tail and a hydrophilic head. The hydrophilic head can be nearly any structure, provided it is neutral and hydrophilic, i.e., polar. An example of a useful hydrophilic head has the following structure:

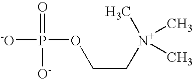

A hydrophobic tail of an unsaturated lipid that is useful herein has the following structure: H₃C—(CH₂)ₙ—X—(CH₂)ₘ—, wherein n and m are each independently 8 to 14, and where the end of the molecule is covalently bound to the hydrophilic head. The hydrophobic tail typically includes one or more structures that permit the polymerization of the tails. For instance, the X portion of the hydrophobic tail can contain at least 2 alkynes, at least 2 alkenes, or a combination thereof. Preferably, the at least 2 alkynes or 2 alkenes are connected head to head, i.e., —C≡C—C≡C—, and —CH═CH—CH═CH—. Such a structure is also referred to as a conjugated alkyne or a conjugated alkene. Preferably, the structure(s) that permit the polymerization of the tails are present in about the middle of the hydrophobic tail. For instance, if the hydrophobic tail has the structure —H₃C—(CH₂)ₙ—X—(CH₂)ₘ—, a conjugated alkyne or alkene can be present at any location in the molecule, preferably between carbons 10 and 17, more preferably between carbons 11 and 16, most preferably between carbons 12 and 15.

A preferred example of an unsaturated lipid is phosphocholine, which has the following structure: 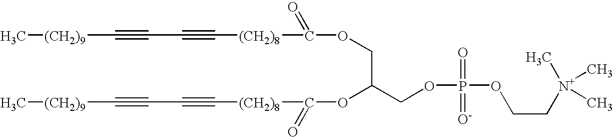
Other examples of polymerizable lipids that can be used to produce polymerized liposomes are disclosed in, for instance, Regen (U.S. Pat. No. 4,485,045), Regen (U.S. Pat. No. 4,808,480), Regen (U.S. Pat. No. 4,594,193), Hasegawa (U.S. Pat. No. 5,160,740), Singh (U.S. Pat. No. 5,466,467), Singh (U.S. Pat. No. 5,366,881), and Regen, in Liposomes: from Biophysics to Therapeutics (Ostro, ed., 1987), Marcel Dekker, N.Y. Additional polymerizable moieties contained within the hydrophobic tail or within the hydrophilic head can be used and have been described and are found in Singh, A., and J. M. Schnur, 1993, “Polymerizable Phospholipids”, in Phospholipids Handbook, Gregor Cevc, ed., Maroel Dekker, New York. Various other polymerizable lipids have been described, having methacrylate, vinylbenzene, diacetylenes, and azidoformaloxy groups within the structure of lipid. Many lipids useful herein (both unsaturated and saturated) are commercially available from, for instance, Avanti Polar Lipids (Alabaster, Ala.). A lipid layer may include more than one type of unsaturated lipid. For instance, the present invention includes liposomes having the unsaturated lipid phosphocholine and other types of unsaturated lipids present in the lipid layer.

In general, useful saturated lipids have the structure H₃C—(CH₂)ₙ—, wherein n is 16 to 28, and where the end of the molecule is covalently bound to a triggering polypeptide. A lipid layer may include more than one type of saturated lipid. Preferably, a saturated lipid does not include any structures that permit the polymerization of the saturated lipid.

The triggering polypeptide is present on the surface of the liposome, bound to the saturated lipid. A trigger polypeptide includes a peptide bond that is cleaved by a protease. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

Typically, a peptide bond that is cleaved by a protease is part of a recognition site that is recognized by a specific protease. In some aspects of the present invention, the recognition site identified by a protease is present on a single linear polypeptide. Examples of proteases that identify a recognition site present on a single linear polypeptide include trypsin, chymotrypsin, and papain. In other aspects of the present invention, a trigger polypeptide includes an amino acid sequence that, upon interaction with two other trigger polypeptides, forms a triple helical conformation. The triple-helical conformation can be made up of three indentical, two identical, or three different trigger polypeptides. The triple helix is typically the structure found in natural type IV collagen; three left-handed poly proline-II-type chains supercoiled in a right-handed manner about a common axis (see Rich and Crick, J. Mol. Biol., 1961, 3, 483-506, and Ramachandran, In: treatise on collagen. Ramachandran, G. N. (Ed.), Academic Press, NY, 1964, 103-183). The trigger polypeptide typically includes an amino acid repeat region. As used herein, an amino acid “repeat region” is (Gly-X—Y)ₘ, where X is proline or a homolog thereof, preferably proline, Y is proline or 4-hydroxyproline or a homolog thereof, preferably proline or 4-hydroxyproline, and m is at least 3. A repeat region in a polypeptide can be GPP, GPO (where O is 4-hydroxyproline), or a combination thereof. This repeat region can be present more than once in the trigger polypeptide, and when it is present more than once the two repeat regions are typically separated by 3 or more amino acids. Without intending to be limiting, it is the repeating sequence that is believed to cause the formation of a triple helix.

An example of a protease that identifies a recognition site present in a triggering polypeptide having a triple helical configuration is a matrix metalloprotease (MMP), a type of extracellular matrix degrading enzyme. There are at least 5 major classes of MMPs: (i) collagenases (MMP1, MMP-8, and MMP-13), (ii) gelatinases (MMP-2 and MMP-9), (iii) stromelysins and stromelysin-like MMPs (MMP-3, MMP-10, and MMP-11), (iv) matrilysins (MMP-7), (v) membrane type MMPs (MMP-14, MMP-15, MMP-16, and MMP-17), and (vi) other MMPs (MMP-20, MMP-23, and MMP-28) (see Fan et al., J. Biochemistry, 1993, 32, 13299-13309, Kramer et al., J. Mol. Biol., 2001, 311, 131-147, and Kramer et al., J. Mol. Biol., 2000, 301, 1191-1205). Preferably, the protease is one that recognizes its cleavage site when the site is present in a triple helical polypeptide. Preferably, the protease is gelatinase-A or gelatinase-B. An example of a gelatinase-A is available at Genbank accession number BC002576, and an example of a gelatinase-B is available at Genbank accession number BC006093. The peptide bond cleaved by gelatinase-A or gelatinase-B is the bond between glycine-leucine and between glycine-isoleucine, thus in some aspects of the present invention the trigger polypeptide includes the amino acid sequence glycine-leucine and/or glycine-isoleucine. Examples of trigger polypeptides that are expected to form a triple helical conformation and include the enzymatic trigger of gelatinase-A and/or gelatinase-B include the following: GPQ GIA GQR (GPO)₃ GG (SEQ ID NO:1), GPQ GIA GQR (GPO)₄ GG (SEQ ID NO:2), GPQ GIA GQR (GPO)₅ GG (SEQ ID NO:3), G (GPO)3 GPQ GIA GQR (GPO)₃ GG (SEQ ID NO:4), G (GPO)₄ GPQ GIA GQR (GPO)₄ GG (SEQ ID NO:5), G (GPO)5 GPQ GIA GQR (GPO)₅ GG (SEQ ID NO:6), GPQ GIA GQR GRV GG (SEQ ID NO:7), GPQ GIA GQR (GPP)₃ GG (SEQ ID NO:8), GPQ GIA GQR (GPP)₄ GG (SEQ ID NO:9), GPQ GIA GQR (GPP)₅ GG (SEQ ID NO:10), G (GPP)₃ GPQ GIA GQR (GPP)₃ GG (SEQ ID NO:11), G (GPP)₄ GPQ GIA GQR (GPP)4 GG (SEQ ID NO:12), G (GPP)₅ GPQ GIA GQR (GPP)₅ GG (SEQ ID NO:13), where O is 4-hydroxyproline, and homologs thereof.

A “homolog” of a polypeptide includes one or more conservative amino acid substitutions, which are selected from other members of the class to which the amino acid belongs. For example, it is well known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide.

For the purposes of this invention, conservative amino acid substitutions are defined to result from exchange of amino acid residues from within one of the following classes of residues: Class I: Ala, Gly, Ser, Thr, and Pro (representing small aliphatic side chains and hydroxyl group side chains); Class II: Cys, Ser, Thr, and Tyr (representing side chains including an —OH or —SH group); Class III: Glu, Asp, Asn, and Gin (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include related amino acids such as 3-Hydroxyproline and 4-Hydroxyproline in Class I; homocysteine in Class II; 2-aminoadipic acid, 2-aminopimelic acid, γ-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoquinoline-3-carboxylic acid, and halogenated tyrosines in Class VI.

Homologs, as that term is used herein, also include modified polypeptides. Modifications of polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

In those aspects where the triggering polypeptide forms a triple helical conformation, the triple helical conformation may be stabilized by the use of an organic scaffold (see, for instance, Goodman et al., Biopolymers (Peptide Science), 1998, 47, 127-142; Jefferson et al., J. Am. Chem. Soc., 1998, 120, 7420-7428; and Kwak et al., J. Am. Chem. Soc., 2002, 124, 14085-14091), transition metal ions (see, for instance, Melacini et al., J. Am. Chem. Soc., 1996, 118, 10359-10364; and Melacini et al, J. Am. Chem. Soc., 1996, 118, 10725-10732), and peptide amphiphiles such a Cys-knot (see, for instance, Muller et al., Biochemistry, 2000, 39, 5111-5116; Ottl et al., FEBS Lett, 1996, 398, 31-36; and Ottl et al., Tetrahedron Lett., 1999, 40, 1487-90) and a Lys-knot (see, for instance, Heidemann et al., Adv. Polym. Sci., 1982, 43, 143-203; Fields et al., Biopolymers, 1993, 33, 1695-1707; and Grab et al., J. Biol. Chem., 1996, 271(21), 12234-12240).

A trigger polypeptide is typically covalently attached to a saturated lipid. Methods for the covalent attachment of two molecules are routine in the art and include, for instance, the use of an amide, ester, or ether bond, streptavidin and biotin (see, for instance, Bally (U.S. Pat. No. 5,171,578)), and activation of a polypeptide with carbodiimide followed by coupling to the activated carboxyl groups (Neurath (U.S. Pat. No. 5,204,096)). Other examples of methods that can be used to covalently bind a polypeptide to a lipid are disclosed in Konigsberg et al. (U.S. Pat. No. 5,258,499).

Optionally, a spacer group is present between the saturated lipid and the triggering polypeptide. A spacer group is nearly any structure that is present between the saturated lipid and the triggering polypeptide, and acts to move the triggering polypeptide further from the surface of the liposome. Many useful spacer groups are commercially available from, for instance, the Aldrich Chemical Company. Generally, a spacer group is hydrophilic, and it can be neutral. Two examples of spacer regions that are useful herein have the following structure:
—CONH—(CH₂CH₂O)_{n′}—,
—(CH₂)_{n″}—NHCO—(CH₂)_{n″′}—,
where n is 1 to 6, and n′, n″, and n″′ are each independently at least 2. A preferred example of a spacer region has the following structure: —CONH—(CH₂CH₂O)₂—(CH₂)₂—NHCO—CH₂—.

The liposomes of the present invention typically have a spherical structure that encapsulates an interior compartment. This interior compartment includes a liquid that is aqueous. The compartment also includes one or more compounds present in the liquid. The compound may be, for instance, a liquid, a solid that is dissolved in the liquid, or a solid that is suspended in the liquid. A compound may be, for example, an organic compound, an inorganic compound, a metal ion, a polypeptide, a non-ribosomal polypeptide, a polyketide, a peptidomimetic, or a polynucleotide. Examples of compounds include, for instance, polynucleotides such as DNA plasmids, positive or negative contrast agents that can be used for imaging such as gadolinium or magnetic particles, fluorescent dyes, chemoattractants, and therapeutic agents, such as chemotherapeutic agents and enzyme inhibitors. A compound may be therapeutic (i.e., able to treat or prevent a disease) or non-therapeutic (i.e., not directed to the treatment or prevention of a disease). Preferably, the liquid includes a pharmaceutically acceptable carrier. “Pharmaceutically acceptable” refers to a diluent, carrier, excipient, salt, etc., that is compatible with the other compounds present in the compartment, and not deleterious to a recipient thereof. The compartment may include a compound that inhibits the activity of the protease that cleaves the trigger polypeptide present on the surface of the liposome. In those aspects of the invention where the trigger polypeptide present on the surface of the liposome is cleaved by gelatinase-A and/or gelatinase-B, an inhibitor of gelatinase-A and/or gelatinase-B activity may be used. Examples of gelatinase-A and gelatinase-B inhibitors are known. An example of such a compound is H-Cys¹-Thr-Thr-His-Trp-Gly-Phe-Thr-Lue-Cys¹⁰-OH (cyclic: 1->10) (SEQ ID NO:14).

Optionally, a liposome of the present invention may include a surface coating of poly(ethyleneglycol) (PEG). Such a surface coating may promote circulation of liposomes (Papahadjopoulos, D. et al., Proc. Natl. Acad. Sci. 88:11460-11464 (1991). Optionally, a liposome of the present invention may include a targeting group. As used herein, a “targeting group” refers to a chemical species that interacts, either directly or indirectly, with the surface of a cell, for instance with a molecule present on the surface of a cell, e.g., a receptor. The interaction can be, for instance, an ionic bond, a hydrogen bond, a Van der Waals force, or a combination thereof. Examples of targeting groups include, for instance, saccharides, polypeptides (including hormones), polynucleotides, fatty acids, and catecholamines. As used herein, the term “saccharide” refers to a single carbohydrate monomer, for instance glucose, or two or more covalently bound carbohydrate monomers, i.e., an oligosaccharide. An oligosaccharide including 4 or more carbohydrate monomers can be linear or branched. Examples of oligosaccharides include lactose, maltose, and mannose. The interaction between the targeting group and a molecule present on the surface of a cell, e.g., a receptor, may, but preferably does not result in the uptake of the targeting group and the covalently attached liposome.

Methods for making polymerized liposomes are known in the art (see, for instance, Singh (U.S. Pat. No. 5,366,881) and Brey et al. (U.S. Pat. No. 6,500,453)). Typically, the lipids are selected to make hybrid liposomes which are less permeable and more stable after polymerization. The criteria for selecting such lipids are known in the art (see, for instance, (Seki et al., Polym. Bull., 13, 489-492 (1985), Takeoka et al., Macromolecules, 24, 1279-1283 (1991), Ringsdorf, In: Physical Chemistry of Biological Interfaces, Baszkin and Norde (Eds), Marcell Dekker, New York, N.Y., pp. 243-282 (2000), Ringsdorf et al., Angew. Chem. Intl. Ed. Engl. 27, 114-158 (1988), Markowitz et al., Diagnostic Biosensor Polymers, American Chemical Society, Washington, D.C., pp. 264-274 (1994), and Singh et al., In: Phospholipids Handbook; Cevc (Ed.), Marcel Dekker, New York, pp. 233-291 (1993)).

The present invention is also directed to compositions including a liposome of the present invention. Such compositions typically include a pharmaceutically acceptable carrier. Additional active compounds can also be incorporated into the compositions.

A composition may be prepared by methods well known in the art of pharmacy. In general, a composition can be formulated to be compatible with its intended route of administration. Examples of routes of administration include perfusion and parenteral, e.g., intravenous, intradermal, subcutaneous, oral (e.g., inhalation), transdermal (topical), transmucosal, and rectal administration. Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials.

Compositions can include sterile aqueous solutions or dispersions and sterile powders for the extemporaneous preparation of sterile solutions or dispersions. For intravenous administration, suitable carriers include, for instance, physiological saline, bacteriostatic water, or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (i.e., a liposome of the present invention) in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent, an edible carrier, or the combination. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can also be prepared in the form of suppositories (e.g., with conventional suppository bases such as cocoa butter and other glycerides) or retention enemas for rectal delivery.

The active compounds may be prepared with carriers that will protect the liposome against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for instance, Alza Corporation and Nova Pharmaceuticals, Inc.

The concentration of liposomes in a composition, e.g., from less than 0.05%, usually at or at least 2-5% to as much as 10 to 30% by weight and will be selected primarily by fluid volumes, viscosities, etc., in accordance with the particular mode of administration selected. Toxicity and therapeutic efficacy of liposomes containing a therapeutic agent can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the methods of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The compositions can be administered one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of a composition containing a liposome of the present invention can include a single treatment or, preferably, can include a series of treatments.

The present invention is further directed to methods for using the liposomes of the present invention. In one aspect, the methods of the present invention include exposing a cell to a compound present in a liposome. In another aspect, the methods of the present invention include treating certain diseases in a subject. The subject is a mammal, preferably a human. As used herein, the term “disease” refers to any deviation from or interruption of the normal structure or function of a part, organ, or system, or combination thereof, of a subject that is manifested by a characteristic symptom or set of symptoms. Diseases include cancers such as, for instance, breast cancer, colorectal cancer, lung cancer, prostate cancer, pancreatic cancer, ovarian cancer, and melanoma. Other diseases include, for instance, gouty arthritis, inflammatory bowel disease (ulcerative colitis), abdominal aortic aneurysms, quiescent Crohn's Disease, glaucoma, and sunlight induced premature skin aging. Typically, whether a subject has a disease, and whether a subject is responding to treatment, is determined by evaluation of symptoms associated with the disease. As used herein, the term “symptom” refers to objective evidence of a disease present in a subject. Symptoms associated with diseases referred to herein and the evaluation of such symptoms are routine and known in the art. Examples of symptoms of cancers include, for instance, the presence and size of tumors and metastatic tumors (i.e., tumors formed by tumor cells from a primary tumor), and the presence and amount of biomarkers. Biomarkers are compounds, typically polypeptides, present in a subject and indicative of the progression of cancer. An example of a biomarker is prostate specific antigen (PSA).

Treatment of a disease can be prophylactic or, alternatively, can be initiated after the development of a disease. Treatment that is prophylactic, for instance, initiated before a subject manifests symptoms of a disease, is referred to herein as treatment of a subject that is “at risk” of developing a disease. An example of a subject that is at risk of developing a disease is a person having a risk factor, such as a genetic marker, that is associated with the disease. Examples of genetic markers indicating a subject has a predisposition to develop certain cancers such as breast, prostate, or colon cancer include alterations in the BRAC1 and/or BRAC2 genes. Another example of a subject at risk of developing a disease is a person having a tumor containing metastatic cells, where such a person is at risk of developing metastatic tumors. Treatment can be performed before, during, or after the occurrence of the diseases described herein. Treatment initiated after the development of a disease may result in decreasing the severity of the symptoms of one of the conditions, or completely removing the symptoms.

The methods typically include administering to a subject at risk of developing a disease or having the disease a composition including an effective amount of a liposome of the present invention, wherein a symptom associated with the disease is decreased. As used herein, an “effective amount” of a composition of the present invention is the amount able to elicit the desired response in the recipient. Whether a liposome of the present invention is expected to function in the methods described herein can be evaluated using ex vivo models and animal models. Such models are known in the art and are generally accepted as representative of disease or methods of treating humans. For instance, the nude mouse model, where human tumor cells are injected into the animal, is commonly accepted as a general model useful for the study of a wide variety of cancers.

The present invention also provides a kit for practicing the methods described herein. The kit includes one or more of the liposomes of the present invention in a suitable packaging material in an amount sufficient for at least one administration. Optionally, other reagents such as buffers and solutions needed to practice the invention are also included. Instructions for use of the packaged liposome(s) are also typically included.

As used herein, the phrase “packaging material” refers to one or more physical structures used to house the contents of the kit. The packaging material is constructed by well known methods, preferably to provide a sterile, contaminant-free environment. The packaging material has a label which indicates that the liposome(s) can be used for the methods described herein. In addition, the packaging material contains instructions indicating how the materials within the kit are employed to practice the methods. As used herein, the term “package” refers to a solid matrix or material such as glass, plastic, paper, foil, and the like, capable of holding within fixed limits the liposome(s). Thus, for example, a package can be a glass vial used to contain appropriate quantities of the liposome(s). “Instructions for use” typically include a tangible expression describing the conditions for use of the liposome(s).

The present invention is illustrated by the following examples. It is to be understood that the particular examples, materials, amounts, and procedures are to be interpreted broadly in accordance with the scope and spirit of the invention as set forth herein.

### EXAMPLE 1

### Triggered Release of Liposomal Contents by Matrix Metalloproteinase-9

This example describes a triggered release methodology of liposomal contents via the enzyme matrix metalloproteinase 9 (MMP-9). To demonstrate this, triple-helical collagen-mimetic peptides were conjugated to stearic acid and the resultant lipopeptides were incorporated into liposomes. These liposomes, when exposed to a catalytic amount of MMP-9, efficiently released the encapsulated fluorescent dye (5-carboxyfluorescein), in the surrounding medium.

Gelatins are the natural substrates for the enzyme MMP-9 (Briknarova et al., J. Biol. Chem., 2001, 276, 27613-27621). For these studies, a mimetic peptide was designed with triple-helical structure, containing the cleavage site for the enzyme MMP-9 (P1, H₂N-GPQGIAGQR(GPO)₄GG-OH (SEQ ID NO:15), where the cleavage site for MMP-9 is underlined). This peptide was conjugated to stearic acid to generate the corresponding lipopeptide LP1 (CH₃(CH₂)₁₆COHN-GPQGIAGQR(GPO)₄GG-OH (SEQ ID NO:16), where the cleavage site for MMP-9 is underlined). Four repeat units of the amino acid triad Gly-Pro-Hyp (GPO) were incorporated in the peptide to impart the triple helical structure (Fiori et al., J. Biol. Chem. 2002, 319, 1235-1242, Gore et al., Langmuir, 2001, 17, 5352-5260, Yu et al., Biochemistry 1999, 38, 1659-1668, and Persikov et al., Biopolymers, 2000, 55, 436-450). P1 and LP1 were synthesized by the solid-phase peptide synthetic protocol, employing the commercially available CLEAR resin as the solid support. The resultant products were purified by the RP-HPLC (C₁₈ column), and characterized by circular dichroism (CD) and mass spectroscopy (MALDI-TOF).

The peptides were synthesized on a Rainin Symphony Quartet automatic peptide synthesizer, using CLEAR resin as the support and HBTU-HOBT as the coupling reagents. Each coupling step was for three hours and repeated twice with 5 fold excess of reagents. Cleavage was performed for 3 hours using a cocktail of CF₃CO₂H-anisole and water (95%-2.5%-2.5%). The crude peptide P1 was purified by RP-HPLC (C₁₈ Vydac column) using a linear gradient of 0-70% acetonitrile in water over 40 minutes. Each solvent contained 0.1% trifluoroacetic acid. For P1, MH⁺ calcd. for C₈₈H₁₃₇N₂₈O₂: 2066.00. Found: 2066.12.

Conjugation with stearic acid was performed using the same procedure as the amino acid coupling with 5 fold excess of reagents. A shaker was used for better mixing of reagents. Cleavage conditions were the same as that for P1. Crude LP1 was purified by RP-HPLC, employing a Vydac diphenyl column. The solvents and the gradient were the same as for P1. For LP1, MH⁺ calcd. for C₁₀₆H₁₇₂N₂₈O₃₁: 2333.27. Found: 2333.32.

CD spectra were recorded on Applied Photophysics PiSTAR instrument using a cell of 0.2 mm pathlength. The concentration of P1 or LP1 was 1 mg/mL in 10 mM phosphate buffer, pH 4.0. The solutions were stored for 12 hours at 4° C. before recording the spectra. For the temperature dependent CD spectra, the sample was equilibrated for 20 minutes at each temperature before recording the spectra.

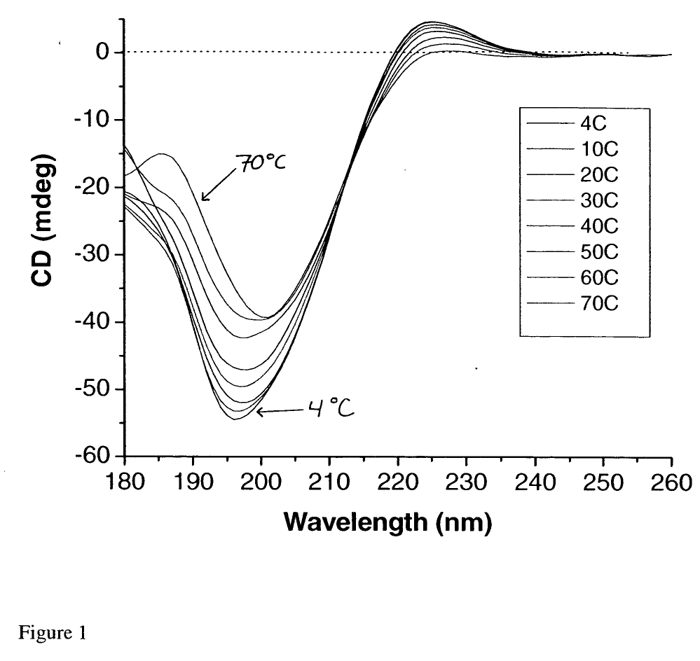

In CD spectra, the triple helical peptides are characterized by strong positive maxima centered at 220-225 nm and an intense negative band located at 196-200 nm (Goodman et al., Biopolymers 1998, 47, 127-142). Both the peptide and lipopeptide showed a positive peaks around 225 nm, and a negative peaks at 200 nm, suggesting their preponderance in the triple helical forms in aqueous solution. The Rpn values for P1 and LP1 were calculated as being equal to 0.06 and 0.11 respectively (for natural collagen, Rpn=0.13) (Feng et al., J. Am. Chem. Soc. 1996, 118, 10351-10358). Temperature dependent CD spectra of LP1 (FIG. 1) showed an isobestic point at 213 nm, suggesting its equilibrium distribution between the two alternative conformational states (e.g., single stranded⇄triple helical). The melting temperature (Tₘ) was calculated (by plotting the CD₂₂₅ as a function of temperature) to be 57° C. Since the peptide P1 did not show any sigmoidal melting curve, no Tₘ could be assigned for this peptide.

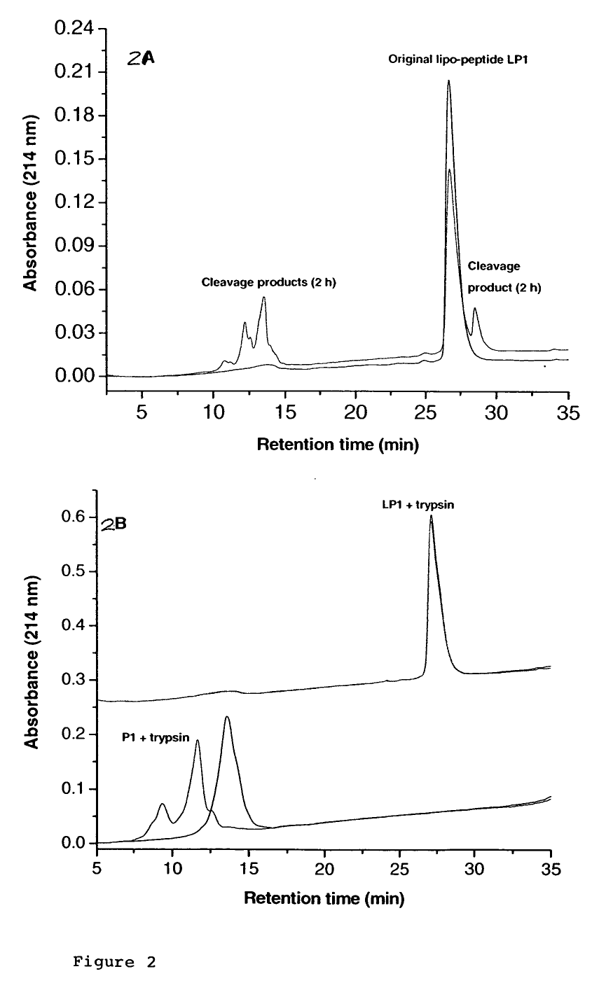

Cleavage studies were performed using the recombinant form of human MMP-9, containing the catalytic and fibronectin domains of the enzyme. The catalytic and fibronectin domains (truncating the hemopexin domains from the full length enzymes) of human MMP-9 were cloned in pET20b vector (Novagen), and over-expressed the enzymes in BL21(DE3) Escherichia coli cells. The expressed proteins were primarily recovered from the inclusion bodies. The inclusion bodies were solubilized in 6 M urea and first subjected to the Q-Sepharose column chromatography. The partially purified proteins were refolded by dilution in 50 mM Tris-HCl buffer, pH 7.8, containing Zn²⁺ and Ca²⁺ ions in the case of gelatinase-A,⁵⁸ but subjected to sequential dialysis (by decreasing concentrations of urea in the above buffer) in the case of gelatinase-B. The refolded gelatinase-A and -B were finally purified by the gelatin-agarose affinity chromatography. The purified MMP-9 showed single band on SDS gel electrophoresis. The yield from 1 liter of bacterial culture was in the range of 20-30 mg. Solutions of P1 (or LP1) were incubated with catalytic amounts of the enzyme and the reaction was stopped at defined intervals by adding trifluoroacetic acid to the reaction mixture. The products were analyzed by RP-HPLC. For the cleavage studies, the conditions were: [P1] or [LP1]=1 mg/mL in 25 mM HEPES buffer, pH 8.0 containing 10 mM CaCl₂; [enzyme]=5 nM; the reaction was stopped by adding 1 uL of CF₃CO₂H. The products were analyzed by RP-HPLC and the conditions are the same as reported for the purification of P1 and LP1. The peptide P1 (Rpn=0.04) was efficiently cleaved by the target enzyme MMP-9 as well as by a non-specific proteolytic enzyme, trypsin (FIG. 2B). However, the lipopeptide LP1 (Rpn=0.11) was partially cleaved by MMP-9 (in 2 hours, FIG. 2A), but was not cleaved at all by trypsin (FIG. 2B). This suggests that unlike trypsin, MMP-9 specifically cleaves the above lipopeptide. Since the amino acid sequences in both P1 and LP1 are the same, the inability of trypsin to cleave the lipopeptide (LP1) is presumably because the enzyme fails to unwind its triple helical structure in order effect the cleavage (Lauer-Fields and Fields, Biol. Chem. 2002, 383, 1095-1105).

Liposomes were prepared (in 25 mM HEPES buffer, pH=8.0) with the synthetic collagen mimetic lipopeptide LP1 (10 mole %) and 1,2-distearoyl-sn-glycero-3-phosphocholine (DSPC) (90 mole %) by following the standard procedure (Roy et al., Org. Lett. 2000, 2, 3067-3070). The liposomes were encapsulated with a self-quenching dye, 5-carboxyfluorescein (Komatsu and Chong, Biochemistry 1998, 37, 107-115). The dye has the excitation maximum at 495 nm, and the emission maximum at 527 nm.

In the lipid bilayers of the liposomes, due to the proximity, the peptide groups of LP1 were expected to form triple helices. The peptides on the outside surface of the liposomes were expected to be recognized and cleaved by MMP-9. After the cleavage, the liposomes were expected to be destabilized, leading to “uncorking” and release of the encapsulated carboxyfluorescein dye. As the dye solution gets diluted upon release, the emission intensity of the solution was found to increase (Komatsu and Chong, Biochemistry 1998, 37, 107-115).

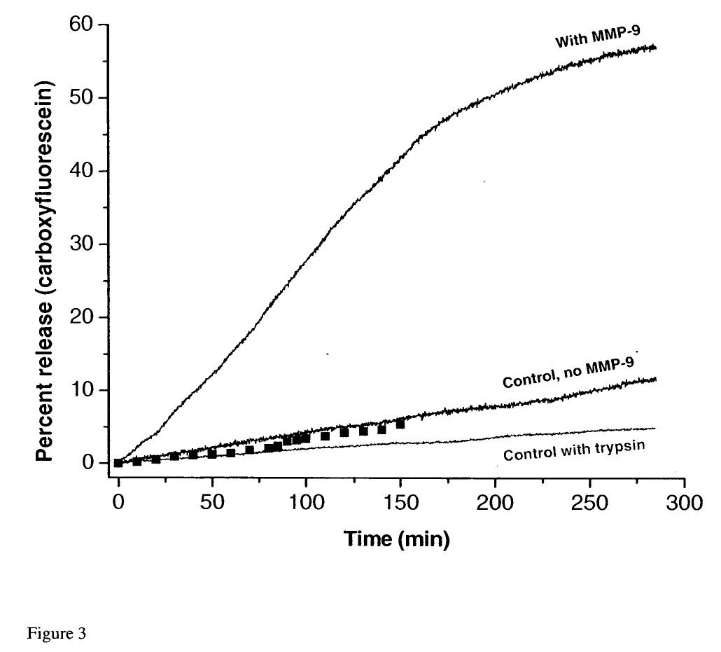

The release of carboxyfluorescein was monitored as a function of time after adding the enzyme, MMP-9 (FIG. 3). For liposome formation, 10% MMPP_{—}4HFA and 90% DSPC (by mole, total lipid concentration of 1 mg/mL in 25 mM in HEPES, 10 mM CaCl₂ at pH 8.0) were dissolved in CHCl₃. A thin film was prepared by evaporating the solvent using a rotary evaporator. The film was placed under high vacuum for 12 h. The film was then hydrated with 150 mM 5-carboxyfluorescein solution (prepared in the same buffer) for an hour at 60° C. followed by sonication for another hour at 60° C. Non encapsulated dye was separated from liposomes through gel filtration chromatography. Before passing through column the osmolarity of the elution buffer (with same composition) was adjusted with liposome solution. This liposome solution was diluted 10 times for the leakage assays. For the leakage assays, 10 μL of MMP-9 (200 nM) was added to a 2 mL of diluted liposome solution in 25 mM HEPES buffer, pH 8.0, containing 10 mM CaCl₂. The emission spectra of the control and liposome+MMP-9 solution were measured. The emission intensity at 520 nm (excitation: 480 nm) was followed as a function of time for 5 h. The conditions for the studies with trypsin were the same as those for MMP-9.

The liposome solution was excited at 480 nm, and the increase in the fluorescent intensity was monitored at 518 nm. There was a time lag of about 5 minutes prior to attainment of a steady-state phase in the fluorescence emission intensity. In five hours, about 55% of the encapsulated dye was found to be released (FIG. 3). In contrast, only 10% of dye was released from the liposomes during this time without any enzyme (FIG. 3). The proteolytic enzyme, trypsin, once again failed to release the dye from the liposome, presumably due it its inability to cleave the liposomal triple helical peptides (FIG. 3). As an additional control, liposomes were prepared from DSPC only. These liposomes did not release any dye when treated with either MMP-9 (FIG. 3, squares) or with trypsin.

These results indicate that the enzyme MMP-9 recognizes the triple helical peptides, protruding from the liposomal surface, and cleaves them. The cleavage results in possible destabilization of the bilayer structure followed by the release of the liposomal contents.

In conclusion, these results demonstrate that the enzyme MMP-9 can be used as a trigger to release liposomal contents. The triple helical peptides act as “baits” for the enzyme. A non-specific proteolytic enzyme (e.g., trypsin) fails to cleave the lipopeptides from the liposomes, and thus no dye release takes place. If the liposomes contain encapsulated inhibitors for MMP-9, this triggered release methodology can be employed to attain the “suicidal” inhibition of the enzyme.

### EXAMPLE 2

### Design of Photocleavable Lipids and their use in Triggered Release of Liposomal Contents

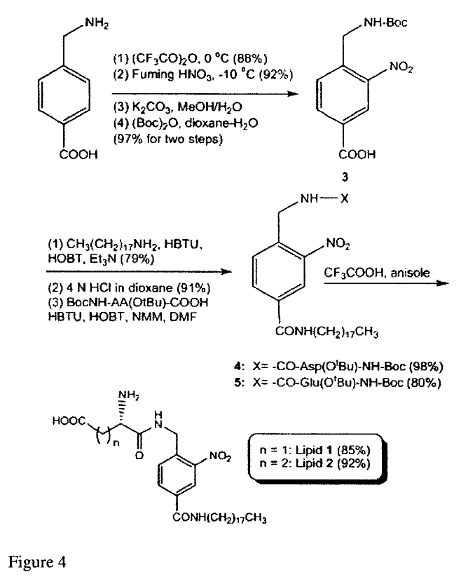

In developing other “triggered” release methodologies, it was noted that o-nitrobenzyl substituted compounds are cleaved by near-UV radiation (Blanc et al., J. Am. Chem. Soc., 2004, 7174-7175; M. C. Pirrung et al., Proc. Natl. Acad. Sci. U.S.A., 2003, 100, 12548-12553; A. Blanc et al., J. Org. Chem., 2003, 68, 1138-1141; K. Schaper et al., Eur. J. Org. Chem., 2002, 1037-1046). There are a few reports of the design of photocleavable lipids (Z. Li et al., Langmuir, 2003, 19, 6381-6391; T. Nagasaki et al., Bioconjugate Chem., 2003, 14, 513-516; Y. Wan et al., J. Am. Chem. Soc., 2002, 124, 5610-5611) (Blanc et al., J. Am. Chem. Soc., 2004, 7174-7175; M. C. Pirrung et al., Proc. Natl. Acad. Sci. U.S.A., 2003, 100, 12548-12553; A. Blanc et al., J. Org. Chem., 2003, 68, 1138-1141; K. Schaper et al., Eur. J. Org. Chem., 2002, 1037-1046). Toward this end, a synthetic scheme was developed for conjugating a C18-amine and selected negatively-charged polar amino acids via the o-nitrobenzyl group (lipids 1 and 2, FIG. 4).

The overall synthesis was accomplished via four easy steps: (i) selective nitration at the o-position of the aminomethyl group of p-aminomethyl benzoic acid, (ii) conjugation of stearylamine at the carboxyl group of compound 3, (iii) removal of the amine protecting group and attachment of the selected amino acids via the a-carboxyl group, (iv) final removal of the protecting groups. A detailed account of the syntheses are given in Example 3.

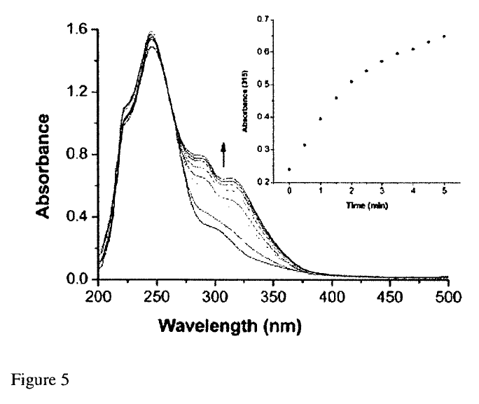

Based on the literature precedent (A. Blanc et al., J. Am. Chem. Soc., 2004, 126, 7174-7175; M. C. Pirrung et al., Proc. Natl. Acad. Sci. U.S.A., 2003, 100, 12548-12553; A. Blanc et al., J. Org. Chem., 2003, 68, 1138-1141; K. Schaper et al., Eur. J. Org. Chem., 2002, 1037-1046), it was anticipated that the o-nitrobenzyl group of the lipids 1 and 2 would be cleaved upon irradiation by UV/visible light in the 320-400 nm region. The photocleavage of the o-nitrobenzyl group proceeds via abstraction of a benzylic hydrogen by the photo-activated nitro group. This is followed by an electron-redistribution to form an aci-nitro form, which finally rearranges to form the o-nitroso benzaldehyde product (Blanc et al., J. Am. Chem. Soc., 2004, 7174-7175; M. C. Pirrung et al., Proc. Natl. Acad. Sci. U.S.A., 2003, 100, 12548-12553; A. Blanc et al., J. Org. Chem., 2003, 68, 1138-1141; K. Schaper et al., Eur. J. Org. Chem., 2002, 1037-1046) (R. Weiboldt et al., J. Org. Chem., 2002, 67, 8827-8831). It is known that the precursor-nitro conjugates exhibit absorption maxima in the range of 250-270 nm (Blanc et al., J. Am. Chem. Soc., 2004, 7174-7175; M. C. Pirrung et al., Proc. Natl. Acad. Sce. U.S.A., 2003, 100, 12548-12553; A. Blanc et al., J. Org. Chem., 2003, 68, 1138-1141; K. Schaper et al., Eur. J. Org. Chem., 2002, 1037-1046) (R. Weiboldt et al., J. Org. Chem., 2002, 67, 8827-8831), the intermediate and final nitroso-derivatives are characterized by the red shift in the corresponding aromatic absorption band by 50-80 nm (R. Weiboldt et al., J. Org. Chem., 2002, 67, 8827-8831) (M. C. Pirrung et al., Proc. Natl. Acad. Sci. U.S.A., 2003, 100, 12548-12553). Hence, the time course of the overall cleavage process of the amphiphilic lipid-amino acid con ugates could be easily probed spectrophotometrically. Since lipids 1 and 2 exhibited similar spectral features, the results are discussed only for lipid 1. FIG. 5 shows the time dependent spectral changes upon irradiation of an ethanolic solution of lipid 1 at 365 nm.

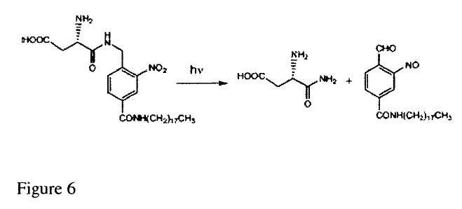

The spectral data of FIG. 5 indicate that the irradiated o-nitrobenzyl group of lipid 1 shows a pronounced absorption peak at 247 nm, with a broad shoulder at 300 nm, and a minor shoulder at 220 nm. As the time of irradiation increases, the intensities of all these peaks increase. However, the shoulder peak of the original (uncleaved) lipid at 300 nm is split into two peaks with absorption maxima at 290 and 315 nm respectively. Of these peaks, the latter is characterized by the formation of a “nitroso” derivative of the cleaved product (R. Weiboldt et al., J. Org. Chem., 2002, 67, 8827-8831). Since the overall spectral changes conformed to clean isosbestic points at 218, 260, and 385 nm, it implied that there were no spectrally distinct intermediates during the course of the overall cleavage process. However, to further probe whether some kinetically significant (albeit spectroscopically undetectable) intermediate was produced during the course of the photocleavage reaction, we analyzed the time slice of the absorption changes at 315 nm. As shown in the inset of FIG. 5, the kinetic profile was best fitted by a single exponential rate equation, with a rate constant of 0.43 min⁻¹, suggesting that the overall cleavage reaction indeed involved a single step. This rate is comparable to reported cleavage rates for the o-nitrobenzyl group under similar irradiation conditions (M. C. Pirrung et al., Proc. Natl. Acad. Sci. U.S.A., 2003, 100, 12548-12553). Based on literature reports,(A. Blanc et al., J. Am. Chem. Sco., 2004, 126, 7174-7175; M. C. Pirrung et al., Proc. Natl. Acad. U.S.A., 2003, 100, 12548-12553; A. Blanc et al., J. Org. Chem., 2003, 68, 1138-1141; K. Schaper et al., Eur. J. Org. Chem., 2002, 67, 8827-8831) (R. Weiboldt et al., J. Org. Chem., 2002, 67, 8827-8831) the structures of the photolysis products for lipid 1 are shown in FIG. 6.

The liposomes were prepared with 1,2-distearoyl-glycero-3-phosphocholine (DSPC, 95% by weight) and 5% of the photocleavable lipid 1 in 50 mM HEPES buffer (pH 5 7.0). The liposomes were characterized by transmission electron microscopy (see Example 3) and the average size of the liposomes was found to be 60-70 nm. A self-quenching hydrophilic dye, 6-carboxyfluorescein, was encapsulated in the liposomes (Liposomes: A Practical Approach, Ed. V. Torchilin and V. Weissig, Oxford University Press, Oxford, 2003). The rate of content release typically depends on the structures of the encapsulated molecules. To facilitate the release, a hydrophilic dye was selected for these studies. The excitation and emission maxima of 6-carboxyfluorescein were determined to be 495 and 518 nm, respectively (see Example 3). Due to the self quenching effect of the above fluorophore at high concentration (H. Komatsu et al., Biochemistry, 1998, 37, 107-115) (the condition which prevails in the lumen of the liposomes due to the local concentration effect), the release of the dye from liposomes (upon uncorking) was expected to proceed in concomitance with the increase in the fluorescence intensity at 518 nm (λex=495 nm). Hence, we could irradiate the 6-carboxyfluorescein encapsulated liposomes at 365 nm (for photocleavage), and monitor their uncorking by measuring the release of the fluorophore at 518 nm (see Example 3).

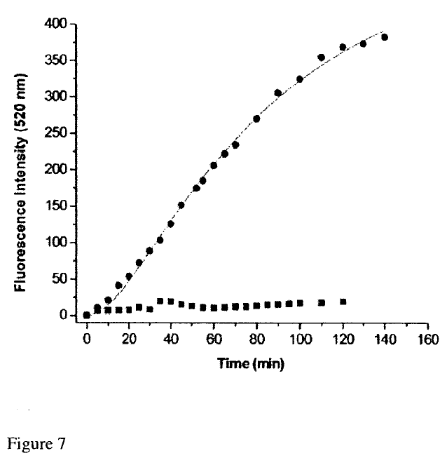

FIG. 7 shows the plot of the increase in the fluorescence intensity at 518 nm as a function of the irradiation (at 365 nm) time. A control experiment was also performed, in which the liposomes were not irradiated (solid squares).

When we attempted to analyze the cleavage data by a single exponential rate equation, the fit was not good. This was not unexpected since the time course of fluorescence increase involves a finite lag phase. Such a kinetic profile could emeroe if the release of the liposome encapsulated fluorophore required some structural adjustments in the liposomal lipid domains. The kinetic data of could be best fitted by a sequential two step kinetic equation (J. W. Moore et al., Kinetics and Mechanism, John Wiley & Sons, Hoboken, N.J., 1981) in the following form [eqn. (1)], with k₁ and k₂ values values of 0.246 and 0.039 min⁻¹, respectively.

$$L - F {⟶}^{{k}_{1}} {L}^{*} - F {⟶}^{{k}_{2}} {L}^{*} + F \\ F - ( L - F ) [ 1 + ( \frac{1}{{k}_{1} - {k}_{2}} ) ( {k}_{2} {ⅇ}^{- {k}_{1} t} - {k}_{1} {ⅇ}^{- {k}_{2} t} ) ] \qquad ( 1 )$$

In eqn. (1), L and F represent liposome and 6-carboxyfluorescein (fluorophore), respectively. L* represents the “intermediary” structure of the liposome, which still harbors the fluorophore in its lumen. The fluorophore is released during the second step. Alternatively, the model mechanism of eqn. (1) can be explained on the basis that the fluorophore exists in the “self-quenched” and “free” states, and the biphasic kinetic profile of FIG. 7 is a result of the transition between such states. Irrespective of the nature of the “species” involved in the overall microscopic pathway, it is clear that the rate constant of photocleavage of lipid 1 (0.43 min−1; FIG. 5) is comparable to that of the first step in eqn. (1).

The similarity of the two rate constants suggests that the first step in the release process is the loss of the hydrophilic head group of the lipid (V. P. Torchilin, Nat. Rev. Drug Discovery, 2005, 4, 145-160). The resultant nitroso benzaldehyde compound (FIG. 6) destabilizes the liposome bilayer. It is possible that lipid reorganization also takes places before the encapsulated dye is released.

Due to ease of the syntheses of o-nitrobenzyl conjugated photocleavable lipids and their abilities to become incorporated in the liposomes, we could demonstrate the feasibility of the photo-induced uncorking of liposomes and release of their contents. The liposomes were found to be stable (in the absence of light) for more than two weeks at 4° C. The rate of contents release is useful for in vivo applications (T. L. Andresen et al., J. Med. Chem., 2004, 47, 1694-1703; J. Davidsen et al., Biochim. Biophys. Acta, 2003, 1609, 95-101; P. Meers, Adv. Drug Delivery Rev., 2001, 53, 265-272) (A. S. L. Derycke et al., Adv. Drug Delivery Rev., 2004, 56, 17-30). Thus, our overall methodology has the potential to find applications in the area of “drug delivery” in biomedical research (V. P. Torchilin, Nat. Rev. Drug Discovery, 2005, 4, 145-160).

### EXAMPLE 3

### Synthesis of Lipids and Dye-Encapsulated Liposomes

Synthesis of Lipids 1 and 2

Materials

Commercial reagents were purchased from either Aldrich or Acros Chemical Co. The protected aminoacids were purchased from Nova Biochem. The Di-Boc protected ornithine was prepared in the lab following standard Boc-protection protocol. Nitric acid (90%) was from Alfa Aesar. All solvents used for reactions were analytical grade and were used without further purification. Melting points were determined on a micro melting point apparatus. ¹H and ¹³C NMR spectra were recorded using 300, 400 or 500 MHz spectrometers using the Varian software. Solvents used for NMR were one of the following: CDCl₃, CD₃OD and DMSO-d₆ with TMS as the internal standard. Elemental analyses were obtained from facilities at Desert Analytics (Tucson, Ariz.). TLC was performed with Adsorbosil plus IP, 20×20 cm plate, 0.25 mm (Altech Associates, Inc.). Chromatography plates were visuialized by either UV light or in an iodine chamber. For drying water-wet compounds, lyophilization (Freeze Dry system/Freezone 4.5; Labconco) was used. Reactions were performed either under an atmosphere of N₂ or using a guard tube. For extractive workups, the organic layer was dried over anhydrous Na₂SO₄, and concentrated ill vacuo.

4-(Boc-aminomethyl)-3-nitrobenzoic acid (3)

Trifluoroacetic anhydride (5.9 mL, 41.34 mmol) was added in small portions to solid 4-(aminomethyl) benzoic acid (2.5 g, 16.54 mmol), while applying external cooling in an ice-bath. Upon completion of addition, the reaction mixture was homogeneous. Stirring was continued at 25° C. for 2 h, and then ice water was added to precipitate the product. The white solid was collected by filtration, washed with water and dried. Yield: 3.63 g (88%), mp: 199-203° C.; ¹H NMR (CDCl₃; 300 MHz): δ 7.91 (d, J=7.8 Hz, 2H), 7.37 (d, J=7.8 Hz, 2H), 4.44 (d, J=5.7 Hz, 2H).

The above compound (3.63 g, 14.68 mmol) was added portion wise over 1 h to 90% nitric acid (20 mL) at −5° C. The mixture was stirred further for 1.5 h at 0° C. and then poured onto ice to precipitate the product. The precipitated solid was filtered, washed with plenty of water to neutral pH, and lyophilized to provide an offwhite solid (3.95 g, 92%). mp: 210° C.; ¹H NMR (CDCl₃; 300 MHz): δ 8.61 (d, J=1.6 Hz, 1H), 8.17 (dd, J=1.6, 8.1 Hz, 1H), 7.507 (d, J=8.1 Hz, 1H), 4.74 (d, J=6.0 Hz, 2H).

A solution of compound 2 (0.68 g, 2.33 mmol) and K₂CO₃ (0.81 g, 5.88 mmol) in MeOH-H2O (1:1, v/v; 16 mL) was maintained at 25° C. for 10 h. The dark yellow solution was concentrated, and DMF (3×10 mL) was added and each time removed in vacuo. The resultant solid was dissolved in dioxane-H₂O (1:1, v/v; 10 mL) to form a solution. Di-tert-butyl dicarbonate (0.77 g, 3.54 mmol) was added, and after 2.5 h, the reaction mixture was concentrated in vacuo. Ether and water were added, and the aqueous phase was washed with ether, brought to pH 3.0 with 10% aqueous citric acid, and extracted with ethyl acetate. The combined organic phases were washed with brine, dried over Na2SO4, and concentrated in vacuo to give the title product as a yellow solid (0.67 g, 97%), m.p. 124-126° C.; ¹H NMR (CDCl₃; 300 MHz): δ 8.74 (d, J=1.6 Hz, 1H), 8.30 (dd, J=1.6, 8.0 Hz, 1H), 7.77 (d, J=8.0 Hz, 1H), 4.65 (d, J=6.3 Hz, 2H), 1.44 (s, 9H).

Compounds 4 and 5

Compound 3 (0.8 g, 2.7 mmol) was dissolved in CHCl₃ (20 mL) and stearic acid (0.71 g, 2.7 mmol), HOBt (0.364 g, 2.7 mmol), HBTU (1.024 g, 2.7 mmol) and Et3N (0.75 mL, 5.4 mmol) were added to the solution. The mixture was stirred at room temperature for 10 h. The reaction mixture was then washed with water, the organic phase dried and solvent was removed in vacuo. The residue was purified by column chromatography (eluant: 5% methanol in chloroform, Rf=0.3) to obtain the pure product as a yellow solid (1.19 g, 81%), mp: 84-86° C.; ¹H NMR (CDCl₃; 300 MHz): δ 8.41 (d, J=1.8 Hz, 1H), 8.00 (dd, J=1.8, 8.1 Hz, 1H), 7.68 (d, J=8.1 Hz, 1H), 6.41 (br, s, NH, 1H), 5.30 (broad s, NH, 1H), 4.59 (d, J=6.6 Hz, 2H), 3.45 (q, J=6.9 Hz, 2H), 1.57-1.65 (m, 2H), 1.42 (s, 9H), 1.24-1.33 (m, 30H), 0.87 (t, J=6.9 Hz, 3H).

To the above compound (1.16 g, 2.12 mmol), was added, 4 N HCl in dioxane (8 mL) and the reaction mixture stirred at room temperature for 3 h. The solvent was then removed under vacuum and water added to the residue. The insoluble white solid was filtered, washed with plenty of water and dried to give the deprotected compound (0.89 g, 94%) as a yellow solid. The compound was carried on to the next step without further purification. 1H NMR (CDCl₃; 300 MHz): δ 8.54 (d, J=1.8 Hz, 1H), 8.03 (dd, J=1.8, 7.5 Hz, 1H), 7.75 (d, J=7.5 Hz, 1H), 4.31 (s, 2H), 3.31 (q, J=6.9 Hz, 2H), 1.53 (m, 2H), 1.1-1.4 (m, 30H), 0.78 (t, J=7 Hz, 3H).

The deprotected compound mentioned in the previous step (0.3 g, 0.67 mmol), Boc-Asp(OtBu)-OH.DCHA salt (0.316 g, 0.67 mmol), HOBT (0.091 g, 0.67 mmol) and HBTU (0.25 g, 0.67 mmol) were taken in DMF (15 mL) and N-methylmorpholine (0.15 mL, 1.34 mmol) was added. The reaction mixture was stirred at room temperature overnight. The solvent was removed in vacuo. Water was added to the residue and extracted with ethyl acetate. The combined organic phases were dried and solvent was removed by rotary evaporation. The crude product was purified by silica gel chromatography (eluant: CHCl₃, Rf=0.2) to yield compound 4 as a white solid (0.480 g, 99%), mp: 90-92° C.; 1H NMR (CDCl3; 500 MHz): δ 8.41 (d, J=1.6 Hz, 1H), 7.98 (dd, J=1.6, 8.0 Hz, 1H), 7.70 (d, J=8.0 Hz, 1H), 4.82-4.70 (m, 2H), 4.51-4.44 (m, 1H), 3.47 (q, J=7 Hz, 2H), 2.96-2.88 (m, 1H), 2.62-2.58 (m, 1H), 1.66-1.58 (m, 2H), 1.45 (s, 9H), 1.42 (s, 9H), 1.40-1.20 (m, 30H), 0.88 (t, J=7.0 Hz, 3H).

In an analogous way, the deprotected compound (0.3 g, 0.67 mmol), Boc-Glu(OtBu)-OH (0.2 g, 0.67 mmol), HOBT (0.09 g, 0.67 mmol) and HBTU (0.25 g, 0.67 mmol) were taken in DMF (15 mL) and Nmethylmorpholine (0.15 mL, 1.34 mmol) was added. The reaction mixture was stirred at room temperature overnight. The work-up procedure was the same as described for compound 4. The crude product was then purified by silica gel chromatography (eluant: CHCl₃, Rf=0.3) to provide the glutamic acid derivative 5 as a yellow solid. Yield: 0.34 g (70%) 1H NMR (CDCl3; 500 MHz): δ 8.42 (d, J=1.6 Hz, 1H), 8.98 (dd, J=1.6 Hz, 8.0 Hz, 1H), 7.71 (d, J=8.0 Hz, 1H), 4.75 (d, J=6 Hz, 2H), 4.15-4.08 (m, 1H), 3.47 (q, J=7 Hz, 2H), 2.43-2.37 (m, 1H), 2.31-2.25 (m, 1H), 2.11-2.02 (m, 1H), 1.95-1.87 (m, 1H), 1.65-1.6 (m, 2H), 1.45 (s, 9H), 1.42 (s, 9H), 1.40-1.20 (m, 30H), 0.88 (t, J=7.0 Hz, 3H).

Lipid 1

To the Boc-Asp(OtBu) derivative (0.40 g, 0.56 mmol), was added 4 mL of trifluoroacetic acid and a drop of anisole. The reaction mixture was stirred at room temperature for two hours. It was then slowly added to water and aqueous NaOH solution was slowly added to neutralize the TFA. The precipitate was collected by filtration, washed with plenty of water and dried to give lipid 1 as a off-white solid (0.27 g, 85%); mp: 154-157° C.; 1H NMR (DMSO-d6; 400 MHz) (without exchangeable protons): δ 8.43 (d, J=1.6 Hz, 1H), 8.10 (dd, J=1.6, 8.0 Hz, 1H), 7.62 (d, J=8.0 Hz, 1H), 4.66-4.56 (m, 2H), 3.96-3.92 (m, 1H), 3.24 (q, J=7 Hz, 2H), 2.75-2.58 (m, 2H), 1.95 (m, 2H) 1.60-1.40 (m, 2H), 1.35-1.17 (m, 30H), 0.82 (t, J=7.0 Hz, 3H); 13CNMR (DMSOd6; 400 MHz) δ 176.05, 171.99, 64.42, 148.52, 136.77, 135.50, 132.54, 130.66, 123.81, 50.71, 37.19, 31.87, 30.38, 29.58-29.23, 27.10, 22.64, 14.45. Anal. Calcd. For C₃₀H₅₀N₄O₆.3CF₃COONa.4H2O: C, 41.46; H, 5.61; N, 5.37. Found: C, 41.25; H, 5.92; N, 5.43.

Lipid 2

To the Boc-Glu(OtBu) derivative (0.26 g, 0.36 mmol), was added 4 mL of trifluoroacetic acid and a drop of anisole. The reaction mixture was stirred at room temperature for 2 h. The work-up procedure was the same as described for lipid 1. Lipid 2 was isolated as a white solid (0.2 g, 99%); 1H NMR (DMSO-d6) (exchangeable protons not reported): δ 8.49 (d, J=1.6 Hz, 1H), 8.14 (dd, J=1.6, 8.0 Hz, 1H), 7.64 (d, J=8.0 Hz, 1H), 4.70-4.62 (m, 2H), 3.92-3.84 (m, 1H), 3.24 (q, J=7 Hz, 2H), 2.36-2.22 (m, 2H), 1.99-1.90 (m, 2H), 1.58-1.40 (m, 2H), 1.35-1.10 (m, 30H), 0.82 (t, J=7.0 Hz, 3H); 13CNMR (CDCl3-CD3OD; 400 MHz): δ 176.02, 4 169.43, 165.68, 147.94, 135.73, 135.38, 132.15, 130.50, 124.08, 52.69, 40.97, 40.47, 31.96, 30.38, 29.73-29.38, 27.13, 26.72, 22.70, 14.03. Anal. Calcd. for C₃₁H₅₂N₄O₆.CF₃COONa.H₂O: C, 55.61; H, 7.85; N, 7.86. Found: C, 55.37; H, 8.07; N, 8.02.

Preparation of Dye-encapsulated Small Liposomes

The photocleavable lipid (0.45 μmoles, 5 mol %) and solid 1,2-distearoyl-sn-glycero-3-phosphocholine (6.716 mg, 8.55 μmoles, 85 mol %) were dissolved in 5 mL of anhydrous chloroform and a very small amount (0.5 mL) of anhydrous methanol in a 25 mL clean, oven-dried round bottomed flask. The organic solvents were then removed in a rotary evaporator under reduced pressure maintaining the bath temperature at 40° C. until at thin and uniform lipid film was formed on the walls of the round bottomed flask. The flask was left on the rotary evaporator for an additional 15 minutes and then allowed to dry in vacuo for at least 20 hours. In another clean dry glass vial, 56 mg (150 μmoles) of 6-carboxyfluorescein was taken in 3 mL of HEPES buffer (25 mM, pH=8.0). The dye was dissolved by first bath-sonicating (to reduce the particle size of the solid granules of the dye) to form a dark brown transparent solution. The thin dry lipid film was then hydrated with the dye solution (3 mL) by rotating slowly in the rotary evaporator bath at 60° C. for 1 hour. The resulting suspension was then subjected to probe sonication (power: 50 W) at 60° C. for 1 hour with constant nitrogen bubbling, to get a clear dark red liposome solution. The total lipid concentration was 9 mM. The osmolarity of the liposome solution was measured with a standard micro osmometer. Sephadex G-50 resin (particle size 50-150μ) was mixed with excess of water to form a gel and the gel was hydrated overnight at 40° C. in the water bath of a regular rotary evaporator. A chromatography column was packed with the gel after cooling to room temperature and equilibriated with 200 mL of water whose osmolarity was made equal to that of the liposome solution by the addition of solid sodium chloride. The liposome solution was then loaded on top of the column and slowly eluted. The liposomes came out first as a yellow nonfluorescent solution and were collected.

Transmission Electron Microscopy of Small Liposomes

Poly-L-lysine (0.5%) was placed on formvar film carbon coated 300 mesh grid for 30 seconds and wicked off with torn filter paper and allowed to dry. Liposome sample was placed on the same grid for 30 seconds and wicked off. The grid was then negatively stained with 0.5% phosphotungstic acid pH adjusted to 7-8 for 1.5 min and wicked off. After allowing the sample to dry, images were obtained using a JEOL 100CX II Transmission Electron Microscope at 80 KeV.

Leakage Experiments from the Liposomes

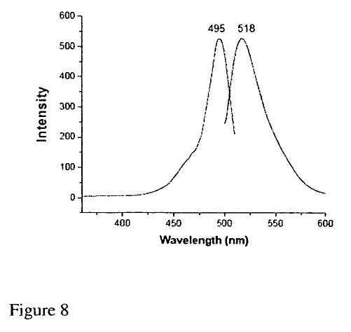

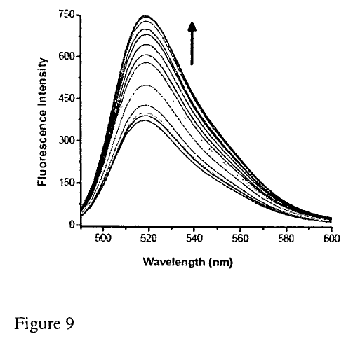

The fluorescence emission spectrum of the dye-encapsulated liposomes was recorded with excitation at 580 nm. The quartz cuvet was then placed under a UV lamp (100 W lamp for the 365 nm irradiation). Every 5 minutes, the cuvet was transferred to the fluorimeter and the emission spectrum was recorded. The intensity of the emission maximum (520 nm) was plotted as a function of time to generate the release curves for the dye-encapsulated liposomes (see FIGS. 8 and 9).

### EXAMPLE 4

### Prevention/attenuation of Metastasis

Various metastatic cancer cell lines are known to overexpress and secrete gelatinase-A and -B in their media (Baker et al., J. Molecular Pathology, 55, 300-304 (2002), and Okada et al., Biochem. Biophys. Res. Commun. 288, 212-216 (2001)). When these enzymes are inhibited by “uncorking” of the hybrid liposomes, the invasion ability of the cancer cells is expected to decrease. The effectiveness of liposome-mediated release of gelatinase inhibitors in attenuating or preventing metastasis is determined using different carcinoma cell lines. The experiments are performed via the invasion assay involving a Modified Boyden Chamber (Plumb et al., Cancer Res., 49, 4435-4440 (1989)). For this assay, 24-well transwell inserts, containing a polyethylene terephthalate (PET) membrane with 8 micrometer pores at the bottom, are used. The surface is coated with Matrigel (Becton-Dickinson), a basement membrane extracted from Engelbreth-Holm-Swarm (EHS) mouse sarcoma cells. Carcinoma cell lines cell lines which are known to secrete gelatinases, HT1080, MCF7, PC3, are used, and NIH3T3 cell lines are used as control. During an invasion assay, about 50,000 cells/insert are seeded on top of the Matrigel membrane and the insert is placed into a well containing a chemoattractant. A number of chemoattractants have been reported, and conditioned media from 3T3 fibroblasts at a 1:2 dilution with PBS is one example. Cells are allowed to invade through the Matrigel and towards the attractant for 8 hours. At this time the Matrigel is removed, and any cells attached to the upper layer will be swabbed away. The membrane containing the invaded cells is washed with PBS, fixed in 75% methanol/25% acetic acid, and stained with 0.4% Crystal violet in methanol/acetic acid. The invaded cells at the bottom surface of the PET membrane are quantified, for instance, as the number of cells per high power field. About ten high power fields are counted per membrane, and the results are averaged. These experiments are performed with liposome encapsulated inhibitors as well as controls with free, unencapsulated inhibtors.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A liposome comprising a trigger polypeptide, a lipid layer, and a compartment surrounded by the lipid layer,
wherein the lipid layer comprises saturated lipids and unsaturated lipids,
wherein a plurality of the saturated lipids comprise a trigger polypeptide,
and wherein three trigger polypeptides form a triple helix.

2. The liposome of claim 1 wherein the unsaturated lipid is polymerized.

3. The liposome of claim 1 wherein the triggering polypeptide comprises an amino acid repeat region.

4. The liposome of claim 1 wherein the amino acid repeat region comprises (GPX)n, wherein X is 4-hydroxyproline, proline, or a homolog thereof, and n is at least 3.

5. The liposome of claim 1 wherein the triggering polypeptide comprises a peptide bond that is cleaved by a gelatinase-A.

6. The liposome of claim 1 wherein the triggering polypeptide comprises a peptide bond that is cleaved by a gelatinase-B.

7. The liposome of claim 1 wherein the compartment comprises a compound.

8. The liposome of claim 7 wherein the compound is an inhibitor of gelatinase-A, gelatinase-B, or the combination thereof.

9. A composition comprising the liposome of claim 1 and a pharmaceutically acceptable carrier.

10. A liposome comprising a trigger polypeptide, a lipid layer, and a compartment surrounded by the lipid layer,
wherein the lipid layer comprises polymerized saturated lipids and unsaturated lipids,
wherein a plurality of the saturated lipids comprise a trigger polypeptide,
wherein the compartment comprises a compound,
and wherein cleavage of a peptide bond within the trigger polypeptide results in release of the compound from the liposome.

11. The liposome of claim 10 wherein three trigger polypeptides form a triple helix.

12. A composition comprising the liposome of claim 10 and a pharmaceutically acceptable carrier.

13. A method for inhibiting activity of an enzyme comprising:
providing a liposome comprising a trigger polypeptide present on the surface of the liposome, a lipid layer, and a compartment surrounded by the lipid layer, wherein the trigger polypeptide comprises a peptide bond that is cleaved by a first enzyme, wherein three trigger polypeptides form a triple helix, and wherein the compartment comprises an inhibitor of a second enzyme;
exposing the liposome to the enzyme, wherein the first enzyme cleaves the peptide bond and the liposome releases the inhibitor, and wherein the inhibitor inhibits the activity of the second enzyme.

14. The method of claim 13 wherein the first and second enzymes are present in vivo.

15. The method of claim 13 wherein first and second enzymes are gelatinase-A or gelatinase-B.

16. A method for treating a disease comprising:
administering to a patient having or at risk of having a disease an effective amount of a composition comprising a liposome, wherein the liposome comprises a targeting polypeptide present on the surface of the liposome, a lipid layer, and a compartment surrounded by the lipid layer, wherein the targeting polypeptide comprises a peptide bond that is cleaved by an enzyme, wherein three trigger polypeptides form a triple helix, and wherein the compartment comprises a compound; and
decreasing a symptom of the disease.

17. The method of claim 16 wherein the compound is an inhibitor of the enzyme.

18. The method of claim 16 wherein the enzyme is gelatinase-A or gelatinasae-B.

19. A method for detecting an enzyme comprising:
administering to a patient an effective amount of a composition comprising a liposome, wherein the liposome comprises a targeting polypeptide present on the surface of the liposome, a lipid layer, and a compartment surrounded by the lipid layer, wherein the targeting polypeptide comprises a peptide bond that is cleaved by an enzyme, wherein three trigger polypeptides form a triple helix, and wherein the compartment comprises an imaging compound; and
detecting the presence of the imaging compound in the patient.
